(19) United States
(12) Patent Application Publication
(10) Pub. No.: US 2015/0126752 A1
(43) Pub. Date: May 7, 2015

(54) PROCESS FOR THE PREPARATION OF 2-C-METHYL-D-RIBONIC-GAMMA-LACTONE

(71) Applicant: BRISTOL-MYERS SQUIBB COMPANY, Princeton, NJ (US)

(72) Inventors: Sarah Evelyn Steinhardt, New Brunswick, NJ (US); Martin D. Eastgate, Trenton, NJ (US)

(21) Appl. No.: 14/407,509

(22) PCT Filed: Jun. 12, 2013
(86) PCT No.: PCT/US2013/045298

§ 371 Date: Dec. 12, 2014

Related U.S. Application Data
(60) Provisional application No. 61/659,153, filed on Jun. 13, 2012.

(51) Int. Cl. C07D 307/33

(52) U.S. Cl. CPC C07D 307/33
USPC 549/314

(57) ABSTRACT

Disclosed is a process to prepare a ribonolactone compound of Formula (I):

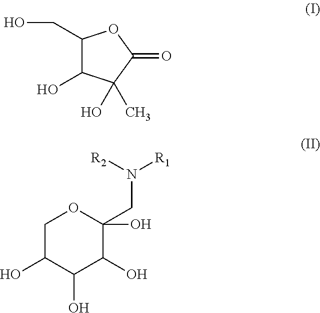

comprising the step of reacting a fructosamine compound of Formula (II): in the presence of a calcium salt and a base in a nonaqueous reaction medium, to provide said ribonolactone compound of Formula (I).

This application claims benefit of U.S. Provisional Application No. 61/659,153, entitled filed on Jun. 13, 2012, which is herein incorporated by reference in its entirety.

### DESCRIPTION

The present invention generally relates to a process for preparing ribonolactone compounds.

Ribonolactone compounds are key intermediates in the preparation of nucleosides and nucleoside derivatives including compounds having antiviral activity. Examples of ribonolactone compounds include 2-C-methyl-D-ribonic-γ-lactone, which is an intermediate in the preparation of the antiviral compound (2S)-2,2-dimethylpropyl 2-((((2R,3R,4R,5R)-5-(2-amino-6-methoxy-9H-purin-9-yl)-3,4-dihydroxy-4-methyltetrahydrofuran-2-yl)methoxy)(naphthalen-1-yloxy)phosphorylamino)propanoate.

U.S. Pat. No. 7,598,373 B2 provides a review of processes to prepare 2-C-methyl-D-ribonic-γ-lactone. This reference also discloses a process for preparing 2-C-methyl-D-ribonic-γ-lactone with a 13.6% yield by reacting D-fructose with calcium oxide in the presence of water.

Hotchkiss et al. (Tetrahedron Letters, 47:315-318 (2006)) discloses processes for preparing methyl ribonolactone from D-glucose using aqueous calcium hydroxide. One disclosed process uses the Amadori reaction to convert D-glucose to its corresponding fructosamine, and then treating the fructosamine with calcium hydroxide in water to provide methyl ribonolactone with an overall yield of 19%.

WO 2007/025304 discloses a process for preparing a saccharinic lactone or acid by reacting a sugar compound with a disubstituted amine to prepare a disubstituted amino sugar intermediate; and then treating the intermediate with base, such as calcium oxide, to produce the saccharinic lactone or acid.

Booth et al. (Tetrahedron Asymmetry 19:2417-2424 (2008)) discloses the synthesis of 2-C-methyl ribono-1,4-lactone from glucose in a one-pot procedure. The procedure includes preparation of a dimethyl fructosamine intermediate and treatment of the intermediate with calcium oxide to provide 2-C-methyl ribono-1,4-lactone with a yield of 27%. The procedure conducted at a larger scale provided a yield of 20%.

Desired is a process for preparing 2-C-methyl-D-ribonic-γ-lactone without use of strong base. Also desired is a process that uses lower levels of calcium containing materials in order to reduce the amount of calcium ion that is removed prior to the isolation of the 2-C-methyl-D-ribonic-γ-lactone. Further, it is desired that the process provides the product with yields equivalent to or better than the yields of existing processes. Also desired is a process for preparing 2-C-methyl-D-ribonic-γ-lactone that is scalable to large scale production in a pilot or manufacturing plant. Furthermore, desired in the art is a process that allows real-time monitoring of the reaction.

The present invention is directed to one or more of these, as well as other important aspects.

### SUMMARY OF THE INVENTION

The present invention fills the foregoing need by providing a process to prepare a ribonolactone compound of Formula (I):

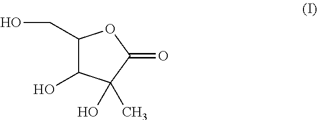

comprising the step of reacting a fructosamine compound of Formula (II):

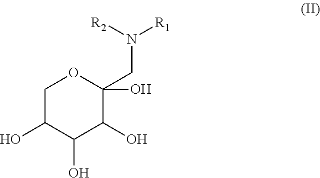

in the presence of a calcium salt in a nonaqueous reaction medium, to provide said ribonolactone compound of Formula (I); wherein R₁ and R₂ are independently selected from C₁₋₆ alkyl, benzyl, allyl, phenyl, or naphthyl, each substituted with zero to 6 substituents independently selected from halogen, —OH, C₁₋₁₂ alkyl, —O(C₁₋₆ alkyl), —NO₂, —NH₂, —N(C₁₋₆ alkyl)₂, —CO₂H, —CO₂(C₁₋₆ alkyl)₂, benzyl, allyl, and/or phenyl; or alternatively, R₁ and R₂ along with the nitrogen atom to which they are attached form a cyclic amine selected from pyrrolidine, piperidine, 1-azacycloheptane, morpholine, or piperazine, wherein said cyclic amine is substituted with zero to 6 substituents independently selected from halogen, —OH, C₁₋₁₂ alkyl, —O(C₁₋₆ alkyl), —NO₂, —NH₂, —N(C₁₋₆ alkyl)₂, —CO₂H, —CO₂(C₁₋₆ alkyl)₂, benzyl, allyl, and/or phenyl.

These and other features of the invention will be set forth in expanded form as the disclosure continues.

### BRIEF DESCRIPTION OF THE DRAWINGS

The invention is illustrated by reference to the accompanying drawings described below.

FIG. 1 shows the concentrations of N-methylbenzyl-fructosamine and methylbenzylamine, as measured by high pressure liquid chromatography, as a function of reaction time in the reaction of N-methylbenzyl-fructosamine in the presence of calcium chloride and sodium methoxide in methanol and tetrahydrofuran to prepare 2-C-methyl-D-ribonic-γ-lactone.

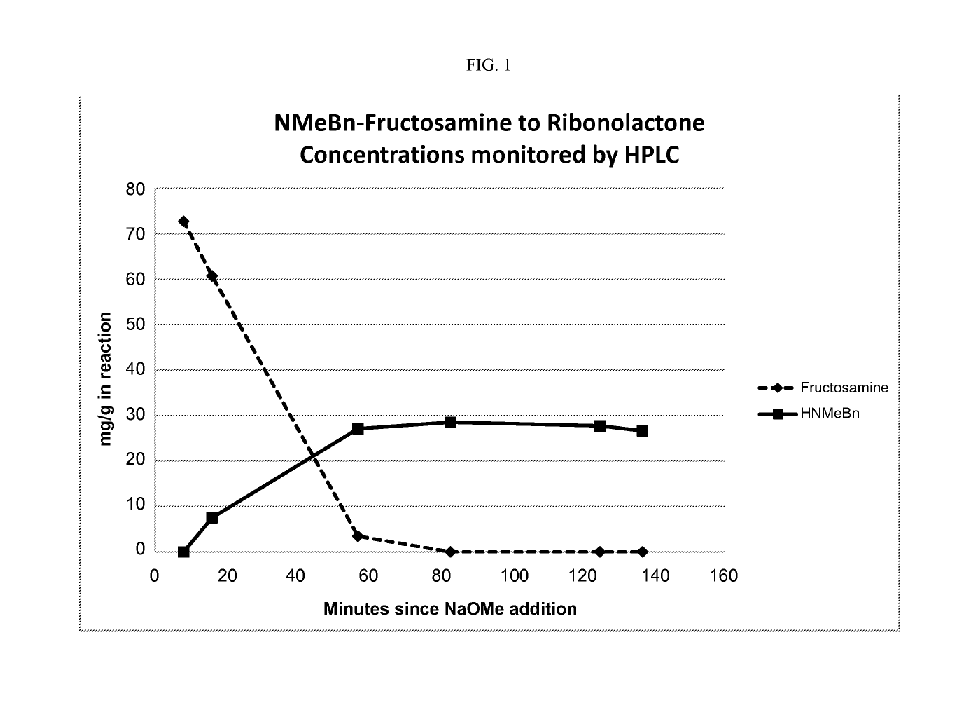

FIG. 2 shows the real time monitoring of the relative concentration of 2-C-methyl-D-ribonic-γ-lactone by infrared absorption at 1783 cm⁻¹ as a function of reaction time in the reaction of N-methylbenzyl-fructosamine in the presence of calcium chloride and sodium methoxide in methanol and tetrahydrofuran to prepare 2-C-methyl-D-ribonic-γ-lactone.

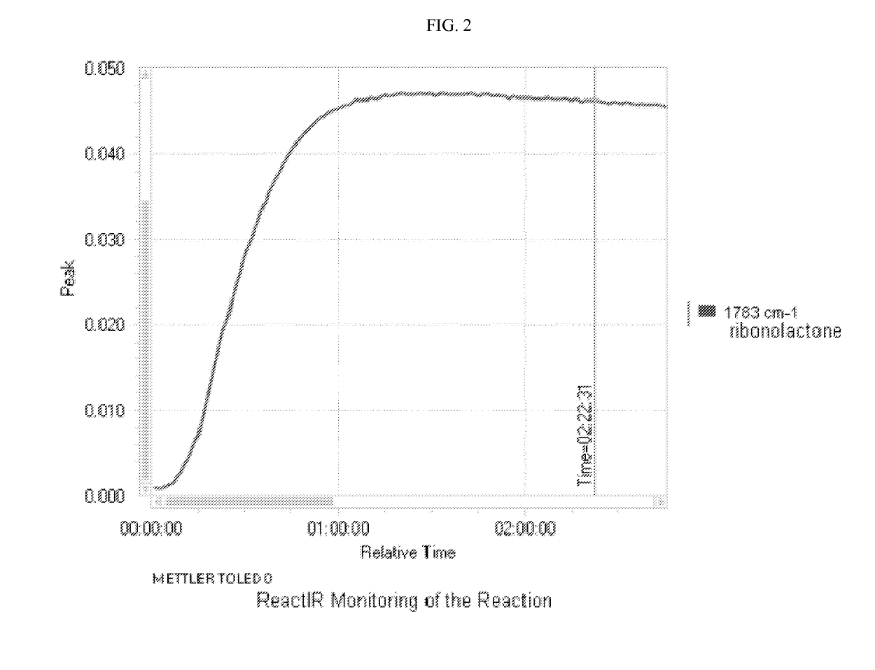

### DETAILED DESCRIPTION

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof. Embodiments identified herein as exemplary or preferred are intended to be illustrative and not limiting.

The definitions set forth herein take precedence over definitions set forth in any patent, patent application, and/or patent application publication incorporated herein by reference.

Listed below are definitions of various terms used to describe the present invention. These definitions apply to the terms as they are used throughout the specification (unless they are otherwise limited in specific instances) either individually or as part of a larger group.

Unless specifically stated otherwise herein, references made in the singular may also include the plural. For example, “a” and “an” may refer to either one, or one or more.

The term “alkyl” and “alk” refer to a straight or branched chain alkane (hydrocarbon) group containing from 1 to 12 carbon atoms, preferably from 1 to 6 carbon atoms, and more preferably from 1 to 4 carbon atoms. Exemplary “alkyl” and/or “alk” groups include, but are not limited to, for example, methyl, ethyl, propyl, isopropyl, n-butyl, t-butyl, pentyl, hexyl, isohexyl, heptyl, octyl, nonyl, decyl, and dodecyl.

The term “lower alkyl” refers to an “alkyl” and/or “alk” group containing from 1 to 4 carbon atoms and preferably from 1 to 2 carbon atoms. When a subscript is used with reference to an alkyl or other group, the subscript refers to the number of carbon atoms the group may contain. For example, the term “C₀-C₄alkyl” includes a bond and an alkyl group containing 1 to 4 carbon atoms, and the term “C₁-C₄alkyl” refers to alkyl groups containing 1 to 4 carbon atoms. Exemplary lower alkyl groups include, but are not limited to, for example, methyl, ethyl, propyl, isopropyl, n-butyl, t-butyl, and isobutyl.

The term “benzyl” refers to a methyl group substituted with a phenyl group.

The terms “halo” and “halogen” refers to F, Cl, Br, or I.

Throughout the specification, groups and substituents thereof may be chosen by one skilled in the field to provide stable moieties and compounds.

The compounds of the present invention are intended to include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

The first aspect of the present invention provides a process for the preparation of a ribonolactone compound of Formula (I):

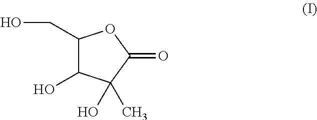

comprising the step of reacting a fructosamine compound of Formula (II):

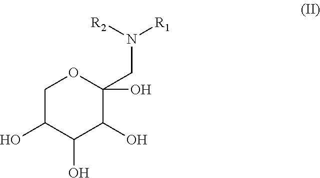

in the presence of a calcium salt in a nonaqueous reaction medium, to provide said ribonolactone compound of Formula (I); wherein R₁ and R₂ are independently selected from C₁₋₆ alkyl, benzyl, allyl, phenyl, or naphthyl, each substituted with zero to 6 substituents independently selected from halogen, —OH, C₁₋₁₂ alkyl, —O(C₁₋₆ alkyl), —NO₂, —NH₂, —N(C₁₋₆ alkyl)₂, —CO₂H, —CO₂(C₁₋₆ alkyl)₂, benzyl, allyl, and/or phenyl; or alternatively, R₁ and R₂ along with the nitrogen atom to which they are attached form a cyclic amine selected from pyrrolidine, piperidine, 1-azacycloheptane, morpholine, or piperazine, wherein said cyclic amine is substituted with zero to 6 substituents independently selected from halogen, —OH, C₁₋₁₂ alkyl, —O(C₁₋₆ alkyl), —NO₂, —NH₂, —N(C₁₋₆ alkyl)₂, —CO₂H, —CO₂(C₁₋₆ alkyl)₂, benzyl, allyl, and/or phenyl. The process optionally employs a base.

In the process of the first aspect of the invention, various solvents, synthesis adjuvants, and reaction conditions can be employed.

Examples of suitable calcium salts include calcium fluoride, calcium chloride, calcium bromide, calcium carbonate, calcium sulfate, calcium sulfite, calcium acetate, calcium chlorate, calcium formate, calcium hydrogen phosphate, calcium nitrate, calcium nitrite, calcium phosphate, calcium dihydrogen phosphate, calcium bromate, calcium iodide, calcium perchlorate, calcium permanganate, calcium oxide, calcium hydroxide, calcium tetrafluoroborate or hydrates thereof. The amount of calcium salt present in the process can be in the range of less than 1 equivalent of calcium based on equivalents of the fructosamine compound of Formula (II). Examples of suitable ranges include 0.1 to 0.9, 0.15 to 0.75, 0.15 to 0.5, and 0.15 to 0.35 equivalents of calcium based on the equivalents of the fructosamine compound of Formula (II).

In one embodiment, the process is conducted in the presence of a calcium salt selected from calcium fluoride, calcium chloride, and calcium bromide. Preferably, the calcium salt is calcium chloride. In one example, the process is conducted in the presence of a calcium salt selected from calcium fluoride, calcium chloride, and calcium bromide, wherein the amount of calcium salt present is in the range of from 0.1 to 0.9, 0.15 to 0.75, 0.15 to 0.5, and 0.15 to 0.35 equivalents of calcium based on the equivalents of the fructosamine compound of Formula (II).

In one embodiment, the process is conducted in the presence of 0.1 to 0.9, 0.15 to 0.75, 0.15 to 0.5, and 0.15 to 0.35 equivalents of calcium chloride based on the equivalents of the fructosamine compound of Formula (II). In one example of this embodiment, the process is conducted in the presence of 0.15 to 0.35 equivalents of calcium chloride, based on the equivalents of the fructosamine compound of Formula (II).

Examples of suitable bases include lithium hydroxide, sodium hydroxide, potassium hydroxide, lithium methoxide, sodium methoxide, and potassium methoxide. The amount of base present in the process can be in the range of less than 1 equivalent of base based on equivalents of the fructosamine compound of Formula (II). Examples of suitable ranges include zero to 0.9, 0.15 to 0.75, 0.15 to 0.5, and 0.15 to 0.35 equivalents of base based on the equivalents of the fructosamine compound of Formula (II).

In one embodiment, the process is conducted in the presence of a base selected from lithium methoxide, sodium methoxide, and potassium methoxide. Preferably, the base is sodium methoxide. In one example, the process is conducted in the presence of a base selected from lithium methoxide, sodium methoxide, and potassium methoxide, wherein the amount of base present is in the range of from zero to 0.9, 0.15 to 0.75, 0.15 to 0.5, and 0.15 to 0.35 equivalents of base based on the equivalents of the fructosamine compound of Formula (II).

In one embodiment, the process is conducted in the absence of a base.

In one embodiment, the process is conducted in the presence of zero to 0.9, 0.15 to 0.75, 0.15 to 0.5, and 0.15 to 0.35 equivalents of sodium methoxide based on the equivalents of the fructosamine compound of Formula (II). In one example of this embodiment, the process is conducted in the presence of 0.15 to 0.35 equivalents of sodium methoxide, based on the equivalents of the fructosamine compound of Formula (II).

The process of the first aspect of the invention is conducted in a nonaqueous reaction medium. As used herein, “nonaqueous reaction medium” refers to the continuous liquid phase in which the various reagents and other synthesis adjuvants employed in the reaction are dissolved, dissociated, dispersed, slurried, or solubilized. The nonaqueous reaction medium comprises predominately one or more organic solvents and is substantially free of water. Examples of suitable organic solvents include, but are not limited to, alcohols, cyclic ethers, dimethylformamide, dimethylacetamide, N-methylpyrollidinone, 1,3-dimethyl-2-imidazolidinone, acetonitrile, dimethylsulfoxide, nitromethane, and mixtures thereof. Examples of suitable alcohols include, but are not limited to, methanol, ethanol, n-propanol, isopropanol, n-butanol, sec-butanol, and tert-butanol. Examples of suitable cyclic ethers include, but are not limited to, tetrahydrofuran, 2-methyl-tetrahydrofuran, tetrahydropyran, and 1,4-dioxane.

As used herein, “substantially free of water” refers to the nonaqueous reaction medium comprising less than 10 weight % water, preferably less than 5 weight % water, and more preferably less than 1 weight % water, based on the weight of the nonaqueous reaction medium. In one embodiment, the nonaqueous reaction medium comprising less than 0.5 weight % water, and more preferably less than 0.1 weight % water, based on the weight of the nonaqueous reaction medium.

In one embodiment, the nonaqueous reaction medium consists essentially of a mixture of methanol and tetrahydrofuran. In one example of this embodiment, the nonaqueous reaction medium consists essentially of a mixture having a ratio of methanol to tetrahydrofuran in the range of from 2:1 to 4:1 (volume/volume).

The process of the first aspect of the invention can be conducted at a range of reaction temperatures. Suitable reaction temperature include temperatures in the range of from 10 to 60° C., temperatures in the range of from 10 to 50° C., and temperatures in the range of from 15 to 45° C.

In one embodiment, the ribonolactone compound of Formula (I) has the structure represented by Formula (Ia):

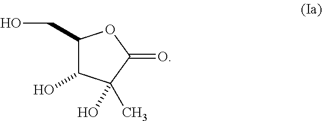

In one embodiment, the fructosamine compound of Formula (II) has the structure represented by Formula (IIa):

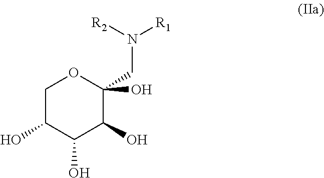

wherein R₁ and R₂ are defined in the first aspect of the invention.

In one embodiment, the fructosamine compound of Formula (II) has the structure represented by Formula (IIb):

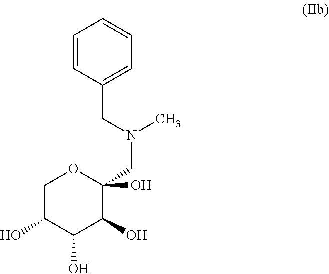 [SMILES: CN(Cc1ccccc1)C[C@@]1(O)OC[C@@H](O)[C@@H](O)[C@@H]1O]

wherein one of R₁ and R₂ is methyl and the other of R₁ and R₂ is benzyl.

In one embodiment, the process for the preparation of a ribonolactone compound of Formula (Ia):

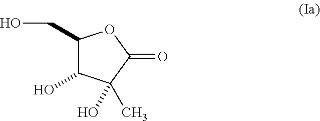

comprising the step of reacting a fructosamine compound of Formula (IIa):

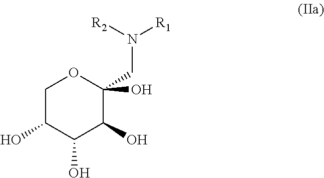

in the presence of a calcium salt and a base selected from lithium methoxide, sodium methoxide, and potassium methoxide in a nonaqueous reaction medium, to provide said ribonolactone compound of Formula (I); wherein R₁ and R₂ are independently selected from C₁₋₆ alkyl, benzyl, allyl, phenyl, or naphthyl, each substituted with zero to 6 substituents independently selected from halogen, —OH, C₁₋₁₂ alkyl, —O(C₁₋₆ alkyl), —NO₂, —NH₂, —N(C₁₋₆ alkyl)₂, —CO₂H, —CO₂(C₁₋₆ alkyl)₂, benzyl, allyl, and/or phenyl; or alternatively, R₁ and R₂ along with the nitrogen atom to which they are attached form a cyclic amine selected from pyrrolidine, piperidine, 1-azacycloheptane, morpholine, or piperazine, wherein said cyclic amine is substituted with zero to 6 substituents independently selected from halogen, —OH, C₁₋₁₂ alkyl, —O(C₁₋₆ alkyl), —NO₂, —NH₂, —N(C₁₋₆ alkyl)₂, —CO₂H, —CO₂(C₁₋₆ alkyl)₂, benzyl, allyl, and/or phenyl.

One embodiment provides a process for the preparation of a ribonolactone compound of Formula (Ia):

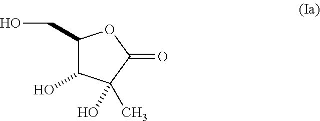

comprising the step of reacting a fructosamine compound of Formula (IIb):

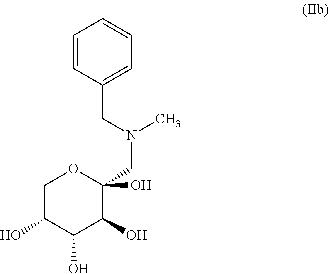 [SMILES: CN(Cc1ccccc1)C[C@@]1(O)OC[C@@H](O)[C@@H](O)[C@@H]1O]

in the presence of calcium chloride and sodium methoxide in a mixture of methanol and tetrahydrofuran, to provide said ribonolactone compound of Formula (Ia). In one example of this embodiment, the fructosamine compound of Formula (IIb) is reacted in the presence of 0.15 to 0.35 equivalents of calcium chloride and 0.15 to 0.35 equivalents of sodium methoxide, each based on equivalents of said fructosamine compound of Formula (IIb); and said mixture has a ratio of methanol to tetrahydrofuran in the range of from 2:1 to 4:1 (volume/volume).

In one embodiment, the process further comprising isolating the ribonolactone compound of Formula (I) by: a) admixing acidic resin particles to said nonaqueous reaction medium; b) removing said acidic resin particles from said nonaqueous reaction medium; and c) crystallizing said ribonolactone compound of Formula (I).

Suitable acidic resin particles include cationic ion exchange resins such as resins having sulfuric acid functional groups (—SO₃H) or carboxylic acid functional group (—CO₂H). Commercially available ion exchange resins include AMBERLYST® resins (Rohm and Haas Co.) such as AMBERLYST®-15 resin, AMBERLYST®-16 resin, AMBERLYST®-31 resin, AMBERLYST®-33 resin, AMBERLYST®-35 resin, AMBERLYST®-36 resin, AMBERLYST®-40 resin, AMBERLYST®-70 resin, AMBERLYST®-121 resin, AMBERLYST®-131 resin, AMBERLITE®-IRC176 resin, AMBERLITE®-IRC747 resin, and AMBERLITE®-IRC748 resin; DOWEX® resins (Dow Co.) such as DOWEX MARATHON® 650C resin, DOWEX MARATHON® C resin, DOWEX MARATHON® C-10 resin, DOWEX MONOSPHERE® C-350 resin, DOWEX MONOSPHERE® C-400 resin, DOWEX® HCR-S/S FF resin, DOWEX MARATHON® MSC resin, DOWEX UPCORE® Mono C-600 resin, DOWEX MONOSPHERE® 650C resin, DOWEX MONOSPHERE® 650HXC resin, DOWEX MONOSPHERE® 650HXC NG resin, DOWEX® HCR-W2 resin, DOWEX® HGR-W2 resin, DOWEX® HGR NG resin, DOWEX MONOSPHERE® 575C NG resin, DOWEX MONOSPHERE® 650C UPW resin, DOWEX MONOSPHERE® 650C NG resin, DOWEX® DR-G8 resin, DOWEX® 88 MB resin, DOWEX® 88 resin, DOWEX MONOSPHERE® 88 resin, DOWEX MONOSPHERE® C-600B resin, DOWEX MONOSPHERE® 575C resin, DOWEX MONOSPHERE® 545C resin, DOWEX MONOSPHERE® 545C NG resin, DOWEX MONOSPHERE® MP-525C resin, DOWEX MONOSPHERE® 750C resin, DOWEX MONOSPHERE® M-31 resin, DOWEX MONOSPHERE® DR-2030 resin, DOWEX UPCORE® Mono MC-575 resin, DOWEX UPCORE® Mono IF-62 resin, DOWEX® M-31 resin, DOWEX® N406 resin, DOWEX® G-26 resin, DOWEX MONOSPHERE® 99Ca/320 resin, DOWEX MONOSPHERE® 99Ca/350 resin, DOWEX MONOSPHERE® 99K/320 resin, DOWEX MONOSPHERE® 99K/350 resin, DOWEX® C-75 NG resin, DOWEX® CM-15 resin, DOWEX® HCR-S resin, DOWEX® HGR resin, and DOWEX® MAC-3 LB resin; AMBERJET® and AMBERLITE® resins (Rohm and Haas Co) such as AMBERJET®1000 resin, AMBERJET® 1300 resin, AMBERJET® 1500 resin, AMBERJET® 1600 resin, AMBERJET® UP1400 resin, AMBERLITE® UP252 resin, AMBERLITE® CR1310Ca resin, AMBERLITE® CR1320Ca resin, AMBERLITE® FPC11 resin, AMBERLITE® FPC14 resin, AMBERLITE® FPC22 resin, AMBERLITE® FPC23 resin, AMBERLITE® SR1L resin, AMBERLITE® IR120 resin, AMBERLITE® IRN77 resin, AMBERLITE® IRN97 resin, AMBERLITE® IRN99 resin, and AMBERLITE® IRP69 resin; and AMBERSEP® 252H resin (Rohm and Haas Co).

Suitable crystallization solvents include, but are not limited to, ethyl acetate, n-propyl acetate, i-propyl acetate, n-butyl acetate, i-butyl acetate, and mixtures with alcoholic solvents; acetone, methyl ethyl ketone, methyl isobutyl ketone, and mixtures; methyl t-butyl ether and mixtures; and mixture thereof.

The fructosamine compound of Formula (II) can be prepared by reacting a unprotected monosaccharide with a secondary amine, for example, using the Amadori reaction. Suitable monosaccharides include D-glucose, L-glucose, D-fructose, L-fructose, D-mannose, L-mannose, D-altrose, L-altrose, D-allose, L-allose, D-psicose, and L-psicose

Processes to prepare the fructosamine compound of Formula (II) are disclosed in Hotchkiss et al., Tetrahedron Lett., 47:315-318 (2006); Booth et al., Tetrahedron: Asymmetry, 19:2417-2424 (2008); and WO 2007/025304.

### Utility

The process of the present invention is useful for preparing ribonolactone compounds of Formula (I). The compounds of Formula (I) are intermediates in the preparation of nucleosides and nucleoside derivatives including compounds having antiviral activity and anticancer activity.

The process of the present invention is useful for preparing the compound of Formula (Ia), which is an intermediate in the synthesis of the anti-hepatitis C compound, (2S)-2,2-dimethylpropyl 2-((((2R,3R,4R,5R)-5-(2-amino-6-methoxy-9H-purin-9-yl)-3,4-dihydroxy-4-methyltetrahydrofuran-2-yl)methoxy)(naphthalen-1-yloxy)phosphorylamino)propanoate, having the structure:

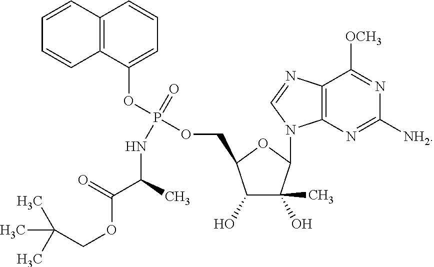 [SMILES: COc1nc(N)nc2c1ncn2C1O[C@H](COP(=O)(N[C@@H](C)C(=O)OCC(C)(C)C)Oc2cccc3ccccc23)[C@@H](O)[C@@]1(C)O]

This compound is disclosed in U.S. Publication No. 2012/052046 as Example 23 along with other phosphoramidate derivatives of guanosine nucleoside compounds useful for treating viral infections.

### EXAMPLES

The invention is further defined in the following Examples. It should be understood that the Examples are given by way of illustration only. From the above discussion and the Examples, one skilled in the art can ascertain the essential characteristics of the invention, and without departing from the spirit and scope thereof, can make various changes and modifications to adapt the invention to various uses and conditions. As a result, the invention is not limited by the illustrative examples set forth hereinbelow, but rather is defined by the claims appended hereto.

### Abbreviations

g gram(s)
h hour(s)
L liter(s)
mL milliliter(s)
mmol millimole(s)
THF tetrahydrofuran
V volume

### Preparation 1

### Synthesis of N-methylbenzylfructosamine from D-Glucose

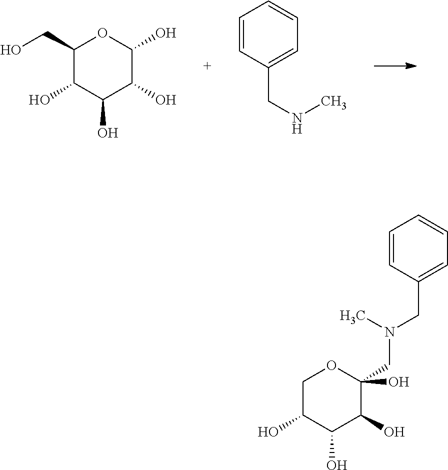

D-glucose (51.59 g, 286.36 mmol) was suspended in ethanol (365 mL, 7 V) at room temperature. To this mixture was added N-methylbenzylamine (37 mL, 286.36 mmol, 1.0 equivalents), followed by acetic acid (15.75 mL, 274.91 mmol, 0.96 equivalents). The mixture was then heated at reflux for 3 h, and cooled to room temperature. Acetone (400 mL, 8 V) was added to the thick slurry, and the solids were then collected by filtration and washed with acetone. The collected solids were dried under vacuum to give 49 g (172.95 mmol, 60% yield) of the fructosamine as an off-white solid. ¹H NMR (500 MHz, DMSO-d₆) δ 7.35-7.28 (m, 5H), 7.27-7.22 (m, 1H), 5.30 (s, 1H), 4.49 (d, J=5.7 Hz, 1H), 4.45 (d, J=5.4 Hz, 1H), 4.38 (d, J=3.2 Hz, 1H), 3.80 (d, J=12.0 Hz, 1H), 3.67-3.64 (m, 2H), 3.64-3.62 (m, 1H), 3.62-3.60 (m, 1H), 3.60-3.53 (m, 3H), 3.41 (d, J=12.0 Hz, 1H), 2.67 (d, J=12.9 Hz, 1H), 2.54 (s, 1H), 2.18 (s, 4H). ¹³C NMR (125.76 MHz, DMSO-d₆) δ 139.0. 128.9, 128.1, 126.8, 98.1, 69.8, 69.0, 63.1, 62.6, 61.9, 43.2.

### Example 1

### Synthesis of 2-Methyl-Ribonolactone from N-Methylbenzylfructosamine

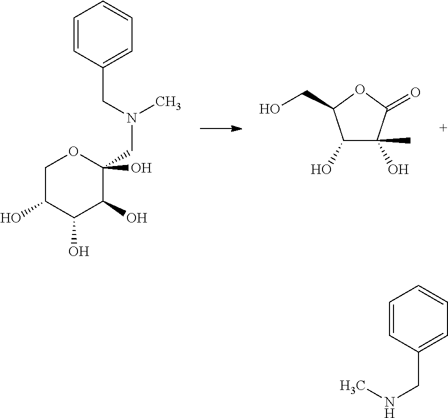

To a slurry of N-methylbenzylfructosamine (200 g, 705.91 mmol) in a mixture of methanol (2.6 L, 13V) and THF (800 mL, 4V) at room temperature was added calcium chloride, anhydrous (19.9 g, 179.31 mmol, 0.25 equivalents), followed by sodium methoxide (25 wt. % solution in methanol, 35 mL, 155.49 mmol, 0.22 equivalents). The reaction mixture was then heated to 40° C. for 16 h. The mixture was then cooled to 20° C. With active cooling, AMBERLYST®-15 resin (dry) was added (600 g, 300 wt. %). The mixture was then stirred at 20° C. for 1 to 2 hours. The AMBERLYST®-15 resin was then removed by filtration, and washed with THF (4 L). The mixture was then concentrated at reflux to approximately 1 L in volume, and ethyl acetate was added (3 L, 15 V). The mixture was then concentrated at reflux to approximately 800 mL in volume, and then cooled to 0° C. The solids were collected by filtration to afford 2-methyl-ribonolactone (66 g, 398.91 mmol, 56.5%) as a very pale grey solid.

### Example 2

### Synthesis of 2-Methyl-Ribonolactone from N-Methylbenzylfructosamine

To a slurry of N-methylbenzylfructosamine (10.12 g, 35.72 mmol) in a mixture of methanol (125 mL, 12 V) and THF (45 mL, 4V) at room temperature was added calcium chloride, anhydrous (0.99 g, 8.9 mmol, 0.25 equivalents), followed by sodium methoxide (25 wt. % solution in methanol, 2.00 mL, 8.9 mmol, 0.25 equivalents). The reaction mixture was then heated to 40° C. for 3 h. The mixture was then cooled to 23° C. With active cooling, AMBERLYST®-15 resin (dry) was added (30.1 g, 300 wt. %). The mixture was then stirred at 23° C. for 1 h. The AMBERLYST®-15 resin was then removed by filtration and washed with THF (150 mL). The mixture was then concentrated under reduced pressure, and ethyl acetate was added (3 L, 15 V). The mixture was then concentrated at reflux to approximately 30 mL in volume, and then cooled to 0° C. The solids were collected by filtration to afford 2-methyl-ribonolactone (3.556 g, 21.93 mmol, 61.4%) as a white powder. ¹H NMR (500 MHz, methanol-d₄) δ 4.27 (ddd, J=7.6, 4.7, 2.5 Hz, 1H), 3.93 (dd, J=12.9, 2.2 Hz, 1H), 3.89 (d, J=7.6 Hz, 1H), 3.70 (dd, J=12.8, 4.6 Hz), 1.39 (s, 3H). ¹³C NMR (125.76 MHz, methanol-d₄) δ 178.2, 84.6, 73.8, 73.7, 61.2, 21.2.

What is claimed is:

1. A process to prepare a ribonolactone compound of Formula (I): 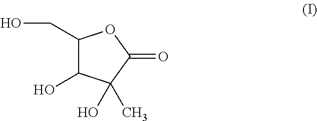
comprising the step of reacting a fructosamine compound of Formula (II): 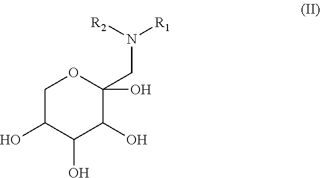
in the presence of a calcium salt in a nonaqueous reaction medium, to provide said ribonolactone compound of Formula (I);
wherein R₁ and R₂ are independently selected from C₁₋₆ alkyl, benzyl, allyl, phenyl, or naphthyl, each substituted with zero to 6 substituents independently selected from halogen, —OH, C₁₋₁₂ alkyl, —O(C₁₋₆ alkyl), —NO₂, —NH₂, —N(C₁₋₆ alkyl)₂, —CO₂H, —CO₂(C₁₋₆ alkyl)₂, benzyl, allyl, and/or phenyl; or alternatively, R₁ and R₂ along with the nitrogen atom to which they are attached form a cyclic amine selected from pyrrolidine, piperidine, 1-azacycloheptane, morpholine, or piperazine, wherein said cyclic amine is substituted with zero to 6 substituents independently selected from halogen, —OH, C₁₋₁₂ alkyl, —O(C₁₋₆ alkyl), —NO₂, —NH₂, —N(C₁₋₆ alkyl)₂, —CO₂H, —CO₂(C₁₋₆ alkyl)₂, benzyl, allyl, and/or phenyl.

2. A process for the preparation of a ribonolactone compound of Formula (Ia): 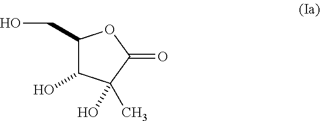
comprising the step of reacting a fructosamine compound of Formula (IIb): 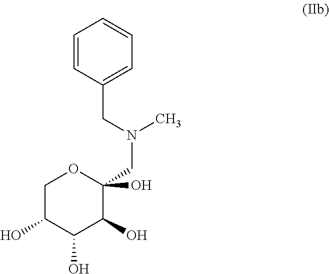 [SMILES: CN(Cc1ccccc1)C[C@@]1(O)OC[C@@H](O)[C@@H](O)[C@@H]1O]
in the presence of calcium chloride and sodium methoxide in a mixture of methanol and tetrahydrofuran, to provide said ribonolactone compound of Formula (Ia).

3. The process according to claim 2 wherein said fructosamine compound of Formula (IIb) is reacted in the presence of 0.15 to 0.35 equivalents of calcium chloride, based on equivalents of said fructosamine compound of Formula (IIb).

4. The process according to claim 2 wherein said fructosamine compound of Formula (IIb) is reacted in the presence of 0.15 to 0.35 equivalents of sodium methoxide, based on equivalents of said fructosamine compound of Formula (IIb).

5. The process according to claim 2 wherein said mixture has a ratio of methanol to tetrahydrofuran in the range of from 2:1 to 4:1 (volume/volume).

6. The process according to claim 2 wherein said fructosamine compound of Formula (IIb) is reacted in the presence of 0.15 to 0.35 equivalents of calcium chloride and 0.15 to 0.35 equivalents of sodium methoxide, each based on equivalents of said fructosamine compound of Formula (IIb); and said mixture has a ratio of methanol to tetrahydrofuran in the range of from 2:1 to 4:1 (volume/volume).

7. The process according to claim 2 further comprising isolating the ribonolactone compound of Formula (I) by:
a) admixing acidic resin particles to said nonaqueous reaction medium;
b) removing said acidic resin particles from said nonaqueous reaction medium; and
c) crystallizing said ribonolactone compound of Formula (I).
